(19) United States
(12) Patent Application Publication
(10) Pub. No.: US 2004/0138293 A1
(43) Pub. Date: Jul. 15, 2004

(54) Pharmaceutical composition made of cannabis extracts

(76) Inventors: Michael Werner, Dornach (DE); Gerhard Schaller, Dornach (DE); Christoph Jaggy, Biel-Benken (CH)

Correspondence Address: Joy I Farber, Hoffmann & Baron, 6900 Jericho Turnpike, Syosset, NY, 11791, US

(21) Appl. No.: 10/469,646

(22) PCT Filed: Feb. 15, 2002
(86) PCT No.: PCT/CH02/00091

(30) Foreign Application Priority Data
Mar. 6, 2001 (CH) 416/01

(51) Int. Cl. A61K 31/353

(52) U.S. Cl. 514/454

(57) ABSTRACT

A pharmacologically active composition which is suitable for use in palliative cancer therapy and as an agent having a muscle-relaxing and/or analgetic effect in neurological diseases. Said composition contains at least 80 wt. %, preferably 90 wt. %, tetrahydrocannabinol (THC) and cannabidiol (CBD), in relation to the overall weight of cannabinoids present therein. The weight ratio of THC to CBD=75:25-20:80, preferably 3:1-1:2, and especially 2:1. Said composition can be used for the production of pharmacologically effective agents which can be used in palliative cancer therapy and in the treatment of neurological diseases.

The present invention relates to a pharmaceutical composition which comprises selected cannabis compounds from the plant Cannabis sativa (herba et flos sicc.). In particular, the composition of the invention comprises the components tetrahydrocannabinol (THC) and cannabidiol (CBD) in selected ratios by weight as described hereinafter. Such compositions show a surprisingly high pharmacological activity in palliative cancer therapy and in the treatment of spasms and for painful muscle stiffness in multiple sclerosis patients. Such compositions furthermore show an excellent pain-relieving effect.

Such components of the cannabis plant Cannabis sativa are substantially known and are also employed in therapy. Thus, for example, Δ⁹-tetrahydrocannabinol (Δ⁹-THC) corresponds to formula (a), and cannabidiol (CBD) corresponds to formula (b).

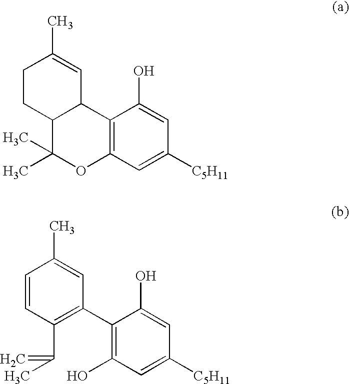

It has now been found that a composition which comprises at least 80% by weight, and preferably at least 90% by weight, of THC and CBD, are calculated from the total weight of the cannabinoids present in the composition, and the THC:CBD ratio by weight is 75:25 to 20:80, preferably 3:1 to 1:2, and in particular 2:1, has a surprisingly high pharmacological activity. In this connection, the stated values for the total weight correspond to the cannabinoids present in the composition, and the values for the THC:CBD ratios by weight correspond to the values calculated or obtained via the peak areas from the corresponding HPLC chromatograms.

The present invention is specified in the claims. In particular, the present invention relates to a pharmacologically effective composition which is suitable for use in palliative cancer therapy and as remedy with a muscle-relaxing and/or pain-relieving effect in neurological disorders, the composition being characterized in that it comprises at least 80% by weight, and preferably at least 90% by weight, of tetrahydrocannabinol (THC) and cannabidiol (CBD), calculated from the total weight of the cannabinoids present in the composition, and the THC:CBD ratio by weight is 75:25 to 20:80, preferably 3:1 to 1:2, and in particular 2:1.

The present invention further relates to a pharmacologically effective solution or suspension of the active ingredients THC and CBD in a suitable solvent or suspension carrier, this solution or this suspension being characterized in that the total weight of THC and CBD in this solution or this suspension is 1% by weight to 25% by weight, preferably 1.5% by weight to 6% by weight, calculated from the total weight of the solution or suspension, the content of THC and CBD, calculated from the total weight of the cannabinoids present in the solution or suspension, being at least 80% by weight, preferably at least 90% by weight, and the THC:CBD ratio by weight being 75:25 to 20:80, preferably 3:1 to 1:2, and in particular about 2:1.

The invention further relates to single-dose administration forms for peroral administration, for example compressed forms such as tablets or coated tablets, and hard gelatin capsules or soft gelatin capsules, preferably soft gelatin capsules which comprise the pharmacologically active composition of the invention.

The present invention relates to the pharmacologically composition of the invention in the form of a plant extract.

The invention further relates to a process for producing a composition of the invention.

The invention further relates to the use of the composition of the invention for producing pharmacologically active remedies for use in palliative cancer therapy and in the treatment of MS-related spasms.

The present invention also relates to the administration of the composition of the invention to patients in palliative cancer therapy and as remedy with muscle-relaxing and/or pain-relieving effect in neurological diseases such as, for example, multiple sclerosis.

The composition of the invention comprises both the compounds produced by a synthetic route and the compounds obtained from plant extracts. The compound tetrahydrocannabinol (THC) can be produced for example by a synthetic route. The synthesis of THC and the control of the byproducts are, however, difficult and the purity of the resulting product is not optimal. The compounds THC and CBD which are used according to the invention are preferably extracted from the plant Cannabis sativa (dried inflorescences and plant, herba et flos sicc.). Operations are always carried out with exclusion of oxygen in each case. The cannabis compounds are moreover extracted from the plant in a manner known per se, for example using low molecular alcohols such as methanol, ethanol, butanol or propanol; acetic esters such as the methyl ester or ethyl ester; ketones, for example acetone; ethers such as methyl ether or ethyl ether; or with low-boiling aliphatic or aromatic or chlorinated hydrocarbons. For example, the cleaned, dried and cut plants (inflorescences, leaves, stalks etc.) are normally treated at the reflux temperature with about three to ten times the amount by weight of the stated solvent or a mixture of such solvents, preferably for at least about one hour, after which the residue is filtered off. The liquid still present in the residue is carefully expelled and added to the filtrate. The solvent is subsequently removed, preferably under vacuum, for example under a pressure of about 80 mbar and at a temperature of about 40-60° C. The resulting extract, which usually results from this process as a honey-like resin, is then heated at a temperature of about 110° C. to about 135° C., preferably at about 120° C., preferably in an autoclave, for about 40 minutes. At this temperature, the compounds which are present as carboxylic acids in the extract are decarboxylated, and the compounds THC and CBD are formed in virtually quantitative yield. The cold product is subsequently taken up preferably in petroleum ether and subjected to a chromatography on silica gel with a suitable mobile phase, for example with a petroleum ether/ethyl acetate mixture. The resulting cannabinoid fraction, which can be detected with the aid of thin-layer chromatography, is subsequently subjected to a chromatographic separation on a hydrophobic silica gel, for example on octadecylsilylated silica gel, preferably using for the chromatography a mobile phase mixture consisting of methanol/water and acetic acid or ethanol/water and acetic acid. This purification results in purified CBD as a first fraction and purified THC as second fraction. The purity of the resulting compounds or active ingredients is determined with the aid of HPLC, and usually a purity of at least 90% by weight based on the total weight of the components present in this fraction is obtained. However, a possible alternative procedure is also to extract the cannabis compounds from the cleaned, dried and cut plant parts in a manner known per se using about three to ten times the amount by weight of an organic solvent which is insoluble in water, that is to say forms a two-phase system with water. Operations are carried out in each case with exclusion of oxygen here too. Suitable solvents are, for example, water-insoluble acetic esters, preferably the methyl or ethyl esters of acetic acid; water-insoluble ethers such as, for example, diethyl ether or ethyl propyl ether; or aliphatic or aromatic or chlorinated hydrocarbons. The organic solvent which contains the extracted cannabis compounds is then filtered and subsequently extracted at least twice with a 2% strength aqueous sodium hydroxide solution, which preferably contains about 20% by weight ethanol. During this, the cannabis compounds which contain a carboxyl group in particular pass into the aqueous/alcoholic phase. The combined aqueous/ethanolic phases are then mixed with a 5% strength sulfuric acid solution so that an acid value (pH) of about 2-4 is produced, after which very low-boiling lipophilic solvent, for example a low-boiling aliphatic or aromatic or chlorinated hydrocarbon, an acetic ester such as, for example, the methyl or ethyl ester of acetic acid or an ether or a mixture of such compounds is used for at least two extractions. The solvent is then removed in vacuo at low temperature. The residue is subsequently subjected to a chromatographic separation of a hydrophobic silica gel, for example on octadecylsilylated silica gel, preferably using for the chromatography a mobile phase mixture consisting of methanol/water and acetic acid or ethanol/water and acetic acid. A UV detector is used for detection at 280 nm. The active ingredient-containing fractions with the natural substances CBD acid and THC acid are obtained. Identification and determination of the purity of the fractions takes place with the aid of HPLC. The extractant (solvent) present in the fractions is then removed in vacuo. The active ingredients or the natural starting substances (i.e. CBD acid and THC acid) are preferably taken up in a lipophilic solvent or a suspension carrier. The active ingredients or active ingredient solutions are subsequently heated at about 110° C. to about 135° C., preferably at about 120° C., preferably in an autoclave, for about 40 minutes with exclusion of oxygen. At this temperature, the compounds which are present in the extract as carboxylic acids are decarboxylated, and the compounds THC and CBD are formed in virtually quantitative yield. The components can be identified using methods known per se, for example using thin-layer chromatography (TLC). High pressure liquid chromatography (HPLC) is advantageously used to determine the purity and content.

Examples of suitable lipophilic solvents or suspension carriers are medium- and/or short-chain triglycerides, medium-chain partial glycerides, polyethoxylated fatty alcohols, polyethoxylated fatty acids, polyoxyethylated fatty acid triglycerides or partial glycerides, esters of fatty acids with low molecular weight alcohols, partial esters of sorbitan with fatty acids, polyethoxylated partial esters of sorbitan with fatty acids, partial esters of sugars or oligomeric sugars with fatty acids, polyethylene glycols, and mixtures of said compounds. Also suitable are mixtures of said compounds with fats, oils and/or waxes or glycols or suspensions in mixtures of lecithins and/or oils and/or waxes.

The extraction process described above usually results in extracts or extract fractions which in each case contain the components tetrahydrocannabinol (THC) or cannabidiol (CBD) in an amount of at least 90% by weight, calculated from the total weight of the cannabinoids present in the extract. The remaining proportions by weight consist of other compounds present in the cannabis plant.

The procedure for producing the mixture of the invention is preferably such that the two solutions containing the active ingredient are mixed together in the appropriate ratio so that a ratio of the active ingredients THC:CBD in the range from 75:25 to 80:20, preferably 3:1 to 1:2, and in particular about 2:1, is obtained.

The total content of tetrahydrocannabinol (THC) and cannabidiol (CBD) in the pharmacologically active solution or suspension is moreover preferably in the range from 1% by weight to 25% by weight, preferably in the range from 1% by weight to 6% % by weight and in particular in the range from 1.5% by weight to 6% by weight, based on the weight of all the ingredients of the solution or of the suspension. On production of tablets or coated tablets, the ratio of the active ingredients THC:CBD remains as stated above, but the concentration thereof based on the total weight of the tablet or coated tablet may be higher.

In the therapeutic use of the composition of the invention in the indication of palliative cancer therapy, on average 5 mg of THC, in a dosage range of 2.5-20 mg of THC, are administered each day as therapeutic dose (this is equivalent to 3.75-120 mg of the composition of the invention, calculated from the dry weight of THC and CBD). In the indication of MS-related spasms, on average 10 mg of THC, in a dosage range of 5-30 mg THC, are administered each day as therapeutic dose (this is equivalent to 7.5-120 mg of the composition of the invention).

The composition of the invention has two main areas of use, namely (i) palliative cancer therapy (loss of appetite/loss of weight, nausea/vomiting, chronic pain and reactive depression) and (ii) muscle-relaxing and/or pain-relieving effect in neurological disorders, especially multiple sclerosis.

The pharmacological effects of the composition of the invention may be assigned to further areas of use as follows:

Appetite-stimulating effect: the appetite-stimulating effect of the composition of the invention can also be utilized therapeutically for anorexia/cachexia of HIV-positive patients (AIDS wasting) and for the postoperative changing of patients (especially those ventilated for a prolonged period) to oral nutrition.

Antiemetic (nausea-inhibiting) effect: the antiemetic effect of the composition of the invention can also be utilized to prevent nausea/vomiting resulting from chemotherapy (with a curative intent) in cancer patients (especially as adjuvant antiemesis during treatment with 5HT3 antagonists) and in antiemetic support therapy of HIV infection/AIDS and hepatitis B.

Analgesic (pain-relieving) effect: the analgesic effect of the composition of the invention can also be utilized therapeutically for chronic pain caused otherwise than by advanced cancer or a neurological disorder, for example for migraine, disorders of the locomotor system and of connective and muscle tissues (arthrosis, arthritis, myopathies), for painful menstruation, for gastrointestinal disorders (e.g. Crohn's disease) and for phantom pain. The analgesic effect of the composition of the invention on neuropathic pain, especially zoster neuralgia, should be particularly emphasized.

Antidepressant (mood-lightening) and anxiolytic (anxiety-reducing) effect: the antidepressant and anxiolytic effect of the composition of the invention can also be utilized for a supportive treatment of other chronic or (now) incurable disorders such as AIDS, paraplegia or chronic rheumatoid arthritis.

Other pharmacological effects of the composition of the invention are a sedative/sleep-promoting effect (sleeplessness), an antiepileptic effect (epilepsies), a bronchiodilating effect (bronchial asthma), a modulation of motor processes (neurological movement disorders such as, for example, dystonias, Tourette syndrome) or a reduction in the intraocular pressure (glaucoma) and an antiinflammatory (inflammation-inhibiting) effect (Crohn's diseases, ulcerative colitis, arthritis, neurodermatitis).

Said pharmacological effects derive in the majority of cases primarily from the THC content in the composition of the invention, but are crucially modulated, modified and thus enhanced in its beneficial effects by the CBD content. Owing to the specifically anxiolytic, antihallucinogenic and antipsychotic effect of CBD it additionally leads to a marked reduction in side effects which may be observed on administration of isolated THC, and thus to improvement of the tolerability of the composition of the invention.

Since the composition of the invention is on the controlled substance list in the narcotics act and may be employed exclusively for clinical studies, until November 2000 it could be used only for the two main areas of use (palliative cancer therapy and muscle spasms associated with multiple sclerosis). A multicenter, randomized, double-blind, placebo-controlled study to compare the composition of the invention with isolated THC in the effect thereof on anorexia/cachexia, nausea and reactive depression, and in the tolerability thereof in cancer patients in the palliative situation is expected to be concluded in autumn 2001, and positive results are to be expected, especially concerning the effect of the composition of the invention compared with the effect of THC.

The following examples illustrate the invention.

### Example 1

### Production of the Individual THC and CBD Fractions

a) 360 grams of dried and cleaned plant material of the plant Cannabis sativa L. (flos et herba sicc., dried inflorescence and plant) were mixed with 3600 grams of ethanol (96% pure) and extracted under reflux at atmospheric pressure for one hour. After cooling, the liquid was expelled from the plant material, and the filtrate was filtered.

b) The solvent was removed from the solution obtained in section a) under reduced pressure at 103 Pa (=100 bar) and at 40° C., and the resulting resinous residue was heated in an autoclave at a temperature of 120° C. for 40 minutes. After cooling, the resin was taken up in petroleum ether, and any residues were filtered off.

Chromatography on silica gel (0.035-0.070 mm, chromatography with a mobile phase mixture of 1% by weight ethyl acetate in petroleum ether) resulted in a cannabinoid fraction which was detected by TLC [silica gel 60 F254 (HPTLC pretreated plate), hexane/diethyl ether 8:2; detection: 125 mg of fast blue B salt in 30 ml of 1 N sodium hydroxide solution, dist. water ad 300 ml, evaluation under daylight]. The isolation of the THC and CBD fractions took place by preparative HPLC on octadecylsilylated silica gel (LiChrospher 100 RP18, 7 μm) using 70% by weight ethanol and 1% by weight glacial acetic acid in water as eluent. Detection took place at 280 nm. Evaporation of the eluent resulted in a THC fraction and a CBD fraction. The purity of the fractions was determined by HPLC. These measurements showed a purity of at least 90% by weight, based on the weight of the dry matter obtained, for both fractions (THC and CBD).

### Example 2

### Production of an Administration Form

The components obtained as in Example 1, section b), were mixed in the ratio of 2.5 mg of THC and 1.25 mg of CBD (each calculated as dry matter). The mixture was taken up with medium-chain partial glycerides. (Imwitor 742 from Huls AG; chemical composition: mono- and diglycerides of C₈-C₁₂-fatty acids), so that a concentration of 10 mg of THC/gram of solution was obtained. Soft gelatin capsules were filled with the solution obtained in this way, using a soft gelatin capsule filling apparatus known per se, resulting in soft gelatin capsules containing 2.5 mg of THC and 1.25 mg of CBD per capsule.

1. A pharmacologically active composition which is suitable for use in palliative cancer therapy and as remedy with a muscle-relaxing and/or pain relieving effect in neurological disorders, the composition being characterized in that it comprises at least 80% by weight, and preferably at least 90% by weight, of tetrahydrocannabinol (THC) and cannabidiol (CBD), calculated from the total weight of the cannabinoids present in the composition, and the THC:CBD ratio by weight is 75:25 to 20:80, preferably 3:1 to 1:2, and in particular 2:1.

2. A composition as claimed in claim 1, characterized in that at least one of the compounds THC or CBD has been produced by a synthetic route.

3. A composition as claimed in claim 1, characterized in that the compounds THC and CBD have been obtained from plant extracts.

4. A composition as claimed in any of claims 1-3, characterized in that the compounds THC and CBD have been obtained as plant extract from the plant Cannabis sativa (dried inflorescences and plant, herba et flos sicc.).

5. A composition as claimed in any of claims 1 to 4, characterized in that the compounds THC and CBD are dissolved in a lipophilic solvent or suspension carrier.

6. A composition as claimed in claim 5, characterized in that the lipophilic solvent or the lipophilic suspension carrier is a medium and/or short-chain triglyceride, a medium-chain partial glyceride, a polyoxyethylated fatty alcohol, a polyoxyethylated fatty acid, a polyoxyethylated fatty acid triglyceride or partial glyceride, an ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, and mixtures of these compounds or mixtures of these compounds with fats, oils and/or waxes or glycols or suspensions in mixtures of lecithins and/or oils and/or waxes.

7. A process for producing a composition as claimed in any of claims 1-6, characterized in that the two solutions containing the active ingredient are mixed in such a way that the ratio by weight of the active ingredients THC:CBD is in the range from 75:25 to 80:20, preferably 3:1 to 1:2, and in particular about 2:1.

8. A pharmacologically active solution or suspension comprising a composition as claimed in any of claims 1-6, characterized in that its total content of tetrahydrocannabinol (THC) and cannabidiol (CBD) is in the range from 1% by weight to 25% by weight, preferably in the range from 1.5% by weight to 6% by weight, based on the weight of all the ingredients.

9. A single-dose administration form for oral administration in compressed form, preferably as tablet or coated tablet, or in the form of hard gelatin capsules or soft gelatin capsules, preferably in the form of soft gelatin capsules, which comprise a composition as claimed in any of claims 1-6.

10. An administration form as claimed in claim 9, characterized in that it comprises a composition as claimed in claims 1-6 in an amount of about 3.75 mg to about 35.5 mg, preferably of about 7.5 mg to about 25 mg (calculated from the dry weight of the total of THC and CBD).

11. The use of a composition as claimed in claims 1-6 or an administration form as claimed in any of claims 8-10 for producing pharmacologically active remedies for use in palliative cancer therapy and in the treatment of neurological disorders.

12. The use of a composition as claimed in claims 1-6 or an administration form as claimed in any of claims 8-10 for producing pharmacologically active remedies with a spasm-lightening effect for the therapeutic treatment in cases of multiple sclerosis, especially MS-related spasms.

13. The use of a composition as claimed in claims 1-6 or an administration form as claimed in any of claims 8-10 for producing pharmacologically active remedies with a pain-relieving effect.

14. The administration of a composition as claimed in claims 1-6 or an administration form as claimed in any of claims 8-10 to patients in palliative cancer therapy and as remedy with muscle-relaxing and/or pain-relieving effect in neurological diseases, especially multiple sclerosis.
